(19) United States
(12) Patent Application Publication
(10) Pub. No.: US 2011/0251651 A1
(43) Pub. Date: Oct. 13, 2011

(54) SCREW

(75) Inventor: YUAN-CHANG HSU, Taichung City (TW)

(73) Assignee: SUNG-PO CHO, Taichung City (TW)

(21) Appl. No.: 12/756,127

(22) Filed: Apr. 7, 2010

(51) Int. Cl. A61B 17/86

(52) U.S. Cl. 606/309; 606/301

(57) ABSTRACT

A screw of the present invention includes a rod defining an axis along its orientation. The axis locates at a center of the rod. The rod has a surface being formed with at least one rod thread. A head is axially extended from the rod, and the head has a surface being formed with at least one head thread. The head is a cone or a pyramid. The largest transversal cross-section of the head is larger than a transversal cross-section of the rod.

### BACKGROUND OF THE INVENTION

1. Field of the Invention

The present invention relates to a screw.

2. Description of the Prior Art

A conventional screw, as disclosed in TW M370019, is adapted to be threaded in a plank or the like for fastening purposes. Some disadvantages arisen when the plank endures serious temperature or humidity change are that the plank might expand or shrink to stuck the screw therein. As a result, the screw is hard to be removed from the plank, and a head thereof may break if excessive force is applied thereon trying to twist the screw.

The present invention is, therefore, arisen to obviate or at least mitigate the above mentioned disadvantages.

### SUMMARY OF THE INVENTION

The main object of the present invention is to provide a screw that obviates or at least mitigates the above mentioned disadvantages.

To achieve the above and other objects, a screw of the present invention includes a rod defining an axis along its orientation. The axis locates at a center of the rod. The rod has a surface being formed with at least one rod thread. A head is axially extended from the rod, and the head has a surface being formed with at least one head thread. The head is a cone or a pyramid. The largest transversal cross-section of the head is larger than a transversal cross-section of the rod.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a perspective drawing showing a screw of the present invention;

FIG. 2 is a side view showing a screw of the present invention;

FIG. 2A is an AA profile of FIG. 2;

FIG. 2B is a profile showing another screw of the present invention;

FIG. 2C is a profile showing yet another screw of the present invention;

FIG. 2D is a DD profile of FIG. 2;

FIG. 2E is a partial enlargement of FIG. 2;

FIG. 3 is a perspective drawing showing yet another screw of the present invention;

FIG. 4 is a side view showing yet another screw of the present invention;

FIG. 4A is an AA profile of FIG. 4.

### DETAILED DESCRIPTION OF THE PREFERRED EMBODIMENTS

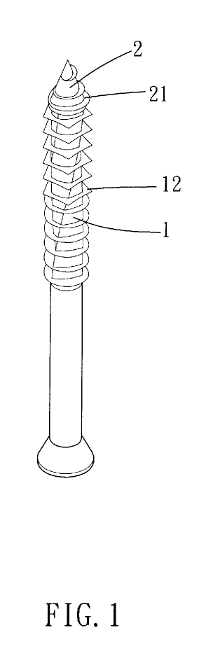

Please refer to FIG. 1. A screw of the present invention is formed with threads to drill an object which is made by wood, plastic or the like material for fastening purposes.

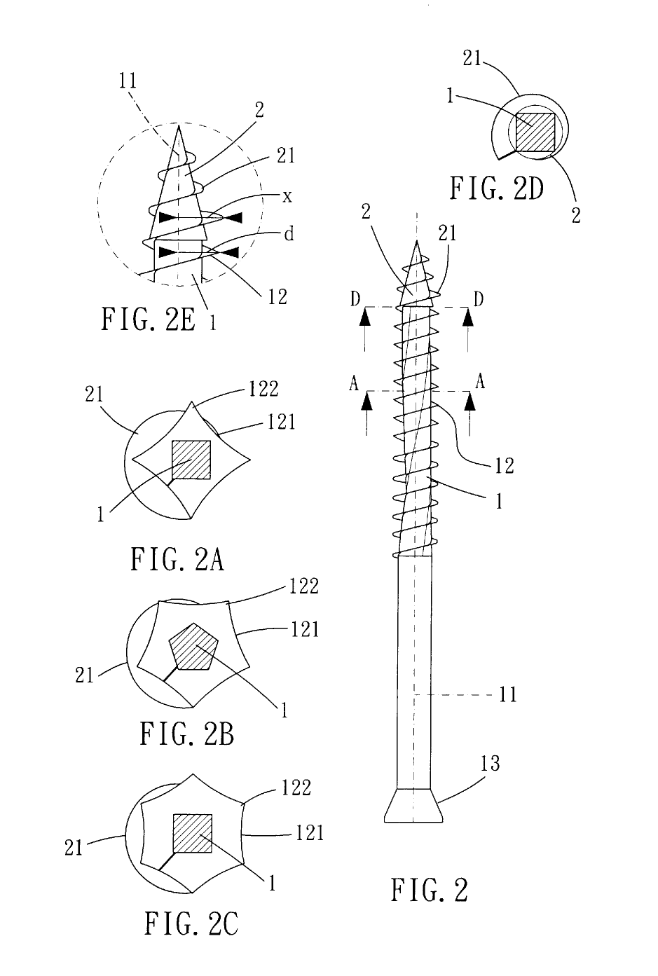

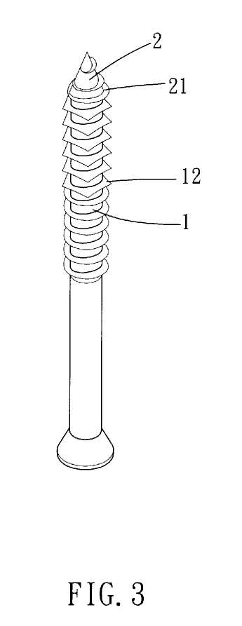

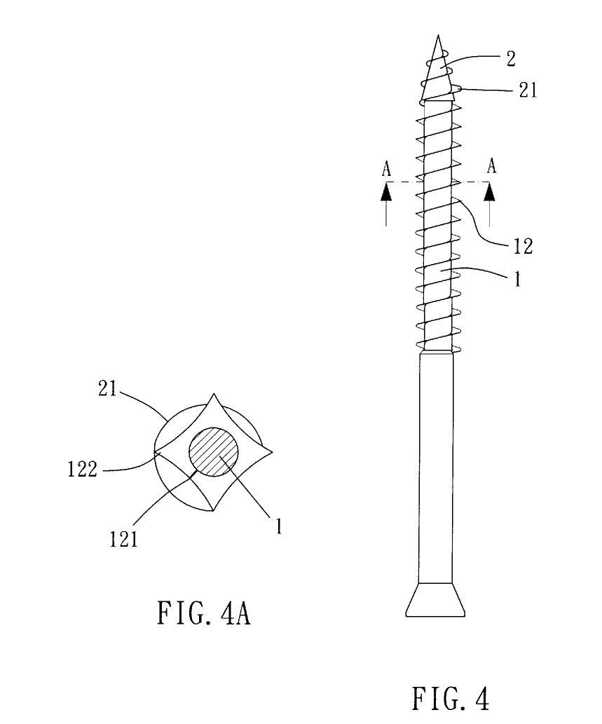

Please refer to FIG. 2. The screw includes a rod 1 which defines an axis 11 along its orientation, and the axis 11 locates at a center of the rod 1. The rod 1 has a surface being formed with at least one rod thread 12, which preferably twists about the axis 11 along a longitudinal direction of the rod 1. Please refer to FIG. 2A. The rod thread 12 has a plurality of radial concaves 121, i.e. the concaves 121 are concave in the radial direction. A protrusive point 122 is formed between each two adjacent concaves 121. More specifically, the concaves 121 may be formed in an arc shape, as shown in FIG. 2A, or other shape suitable. These concaves 121 can be formed on at least a part of the rod thread. As shown in FIG. 2A, four complete concaves 121 can be seen in a rod thread circular section. However, the number of concaves in a rod thread circular section can be varied, as shown in FIG. 2B and FIG. 2C. As shown in FIG. 2A, the transversal cross-section of the rod is rectangular. Yet the cross-section of the rod can be pentagonal, as shown in FIG. 2B, or other non-circular shape. Such non-cylindrical rod may has surface(s) twists about the axis along a longitudinal direction thereof. In other embodiment, e.g. as shown in FIG. 3, FIG. 4 and FIG. 4A, the transversal cross-section of the rod may also be circular.

Please refer to FIG. 2 again. The rod has a first end and a second end. A driving portion 13 is formed on the first end of the rod for a tool to drive therewith. A head 2 is axially extended from the second end of the rod, and the head 2 is a cone or pyramid. The head 2 has a largest transversal cross-section which is larger than that of the rod 1. If the head is a cone, the cross-section thereof is a circle. Thus the cross-section of the head can completely enclose the cross-section of the rod therein as the screw is viewed axially. On the other hand, if the head is a pyramid, the cross-section thereof is a polygon. Thus the definition of the cross-section of the head means a circumcircle of the cross-section of the head. Such circumcircle can completely enclose the cross-section of the rod as the screw is viewed axially. The head 2 has a surface being formed with at least one head thread 21. A maximum transversal distance from the head thread 21 to the axis 11 is bigger than that from the rod thread 12 to the axis 11. More specifically, the transversal distance from the head thread 21 to the axis 11 is x, and that from the rod thread 12 to the axis 11 is d. The maximum x is preferably bigger than d, so that a part of the head thread can be more protrusive than the rod thread in the radial direction.

As such, the head 2 can be used to drill an object and form a hole on the object. Because the head 2 has bigger transversal cross-section, the hole drilled by the head is slightly bigger than the rod. Thus the rod can be easily threaded into the object. Moreover, even when the object endures serious climate, e.g. temperature or humidity, change and expands or shrinks, the screw of the present invention can still be disengaged from the slightly-bigger hole with littler effort. Similar situations occur when part of the head thread is wider than the rod thread. Besides, the rod thread of the present invention may be further formed with concaves. As such, the friction between the rod thread and the object is reduced, so that the screw can be further easily threaded into or out of the object.

What is claimed is:

1. A screw, comprising a rod defining an axis along its orientation, the axis locating at a center of the rod, the rod having a surface being formed with at least one rod thread, a head being axially extended from the rod, the head having a surface being formed with at least one head thread, the head being a cone or a pyramid, a largest transversal cross-section of the head being larger than a transversal cross-section of the rod.

2. The screw of claim 1, wherein a maximum transversal distance from the head thread to the axis is bigger than that from the rod thread to the axis.

3. The screw of claim 1, wherein the transversal cross-section of the rod is non-circular, the surface of the rod twists about the axis along a longitudinal direction of the rod.

4. The screw of claim 1, wherein the rod thread twists about the axis along a longitudinal direction of the rod, the rod thread has a plurality of radial concaves, a protrusive points is formed between each two adjacent concaves.

5. The screw of claim 3, wherein the rod thread twists about the axis along a longitudinal direction of the rod, the rod thread has a plurality of radial concaves, a protrusive points is formed between each two adjacent concaves.
